(19) United States
(12) Patent Application Publication
(10) Pub. No.: US 2010/0076086 A1
(43) Pub. Date: Mar. 25, 2010

(54) PROCESS FOR THE PREPARATION OF O-DESMETHYL VENLAFAXINE

(75) Inventor: Vinayak G. Gore, Maharashtra (IN)

Correspondence Address: FULBRIGHT & JAWORSKI L.L.P.;Attn: MN IP Docket, 600 Congress Avenue, Suite 2400, Austin, TX, 78701, US

(73) Assignee: Merck Development Centre Private Limited Plot 1 A/2, Maharashtra (IN)

(21) Appl. No.: 12/522,318

(22) PCT Filed: Jan. 31, 2008
(86) PCT No.: PCT/GB08/50065

§ 371 Date: Jul. 7, 2009

(30) Foreign Application Priority Data
Jan. 31, 2007 (IN) 184/MUM/2007

(51) Int. Cl. A61K 31/137; C07C 233/02; C07C 215/42; A61P 25/18; A61P 3/04

(52) U.S. Cl. 514/649; 564/182; 564/360

(57) ABSTRACT

The present invention relates to a novel process for the preparation of O-desmethyl venlafaxine (ODV).

### CROSS-REFERENCE TO RELATED APPLICATION(S)

This application is a Section 371 National Stage Application of International No. PCT/GB2008/050065, filed 31 Jan. 2008 and published as WO 2008/093142 A1 on 7 Aug. 2008, which claims priority from the India Application 184/mum/2007, filed 31 Jan. 2007, the contents of which are incorporated herein in their entirety for all purposes.

### FIELD OF THE INVENTION

The present invention relates to a novel process for the preparation of O-desmethyl venlafaxine (ODV).

### BACKGROUND OF THE INVENTION

O-Desmethyl venlafaxine (ODV) (I), chemically named 1-[1-(4-hydroxyphenyl)-2-(dimethylamino)ethyl]cyclohexanol, is a major metabolite of venlafaxine. ODV is known to inhibit norepinephrine and serotonin uptake and to have antidepressant activity. It has been further reported that oral administration of ODV succinate, in particular in sustained release form, results in a lower incidence of nausea, vomiting, diarrhoea, abdominal pain, headache, vaso-vagal malaise and/or trismus than oral administration of venlafaxine. ODV is known to be effective in treating patients suffering from depression, anxiety and panic disorder.

Various patents describe processes for the preparation of ODV free base, which can be converted into a desired pharmaceutically acceptable salt. Such prior art processes to obtain ODV are disclosed in U.S. Pat. Nos. 4,535,186, 4,761,501, 6,673,838, WO 03/48104 and WO 00/59851. The process to obtain ODV disclosed in U.S. Pat. No. 4,535,186 and U.S. Pat. No. 4,761,501 is disclosed in the scheme in FIG. 1.

However, the processes disclosed in the prior art suffer from several disadvantages such as moderate to low yields; obtaining ODV in an impure state; using expensive, toxic and/or hazardous reagents, such as oxalyl chloride, lithium aluminium hydride, concentrated sulphuric acid and n-butyl lithium, which are not recommended to be used on commercial scale; high temperatures to demethylate venlafaxine; and lengthy processes involving the isolation of intermediates.

Consequently there is a need for an improved process for the preparation of ODV or its pharmaceutically acceptable salts which is a relatively safe, short, economical, high yielding and environmentally friendly process which avoids the use of potentially hazardous reagents.

### SUMMARY OF THE INVENTION

The present invention provides a novel process for the preparation of highly pure ODV free base. The process can be used easily for commercial production with a high degree of consistency in quality and yield. Subsequently the ODV free base prepared by the process of the present invention can be converted into any suitable pharmaceutically acceptable salt, such as the succinate or fumarate salt, for dosage form preparation. In addition, the present invention offers a simple work-up procedure with improved yield and quality and with minimum contamination by process impurities.

The present invention also provides a process for the preparation of ODV free base with improved yields, which is amenable to large scale production wherein reaction conditions can be easily controlled. Additionally it is desirable that the product should be in a form that is readily filtered and easily dried.

According to a first aspect of the invention there is provided a process for the preparation of acetamide (VII), comprising reacting acid (VI) with thionyl chloride and dimethylamine or a salt thereof, wherein the group X is any group capable of being converted into a hydroxyl group. The group X is preferably chosen from an alkoxy group such as methoxy, ethoxy or t-butyloxy; an arylalkoxy group such as benzyloxy or p-methoxybenzyloxy; a halide group such as chloro, bromo or iodo; or an acyloxy group such as methoxycarbonyl, ethoxycarbonyl or benzoyl. The group X is preferably benzyloxy. The dimethylamine is preferably used as the hydrochloride salt. The reaction is preferably carried out in an organic solvent such as dichloromethane. The reaction is preferably carried out in the presence of a base such as triethylamine. The conversion of the substituted phenyl acetic acid (VI) to the corresponding acetamide (VII) according to the first aspect of the invention is preferably carried out without isolating the intermediate acid chloride. Preferably acetamide (VII) is obtained from acid (VI) in a yield of 85%, 90%, 95% or more.

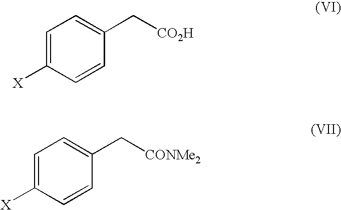

According to a second aspect of the invention there is provided a process for the preparation of O-desmethylvenlafaxine (I) or a pharmaceutically acceptable salt thereof, preferably the succinate or fumarate salt, wherein the process comprises a step according to the first aspect of the invention.

According to a third aspect of the invention there is provided a process for the preparation of amido cyclohexanol (VIII), comprising reacting acetamide (VII) with a base and cyclohexanone, wherein the group X is any group capable of being converted into a hydroxyl group. The base is preferably strong, non-nucleophilic, bulky and/or sterically hindered. Preferably the base is less nucleophilic and/or more bulky and/or more sterically hindered than n-BuLi and/or LDA. Preferred bases are lithium hexamethyl disilazide (LHMDS), potassium tertiary butoxide, NaNH₂, DBU (1,8-diazabicyclo[5.4.0]undec-7-ene), and DBN (1,5-diazabicyclo[4.3.0]non-5-ene). The most preferred base is lithium hexamethyl disilazide (LHMDS). The group X is preferably chosen from an alkoxy group such as methoxy, ethoxy or t-butyloxy; an arylalkoxy group such as benzyloxy or p-methoxybenzyloxy; a halide group such as chloro, bromo or iodo; or an acyloxy group such as methoxycarbonyl, ethoxycarbonyl or benzoyl. The group X is preferably benzyloxy. The reaction is preferably carried out at low temperature. The reaction temperature is preferably lower than −20° C., more preferably lower than −40° C., and most preferably lower than −60° C. The reaction is preferably carried out in a preferably dry organic solvent such as tetrahydrofuran, toluene, diisopropyl ether, methyl t-butyl ether or diethyl ether. The most preferred solvent is tetrahydrofuran. Preferably amido cyclohexanol (VIII) is obtained from acetamide (VII) in a yield of 50%, 60%, 70%, 80%, 82%, 85%, 90% or more.

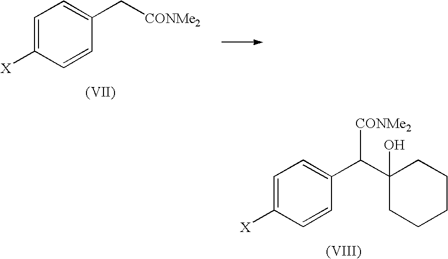

According to a fourth aspect of the invention there is provided a process for the preparation of O-desmethylvenlafaxine (I) or a pharmaceutically acceptable salt thereof, preferably the succinate or fumarate salt, wherein the process comprises a step according to the third aspect of the invention.

According to a fifth aspect of the invention there is provided a process for the preparation of amino cyclohexanol (IX), comprising reducing amido cyclohexanol (VIII) with a reducing agent, wherein the group X is any group capable of being converted into a hydroxyl group. Preferred reducing agents are a borane complex (such as borane-tetrahydrofuran complex, methyl 6-morpholinohexyl sulphide borane complex, borane 1,2-bis(tert-butylthio)ethane complex, borane 4-methylmorpholine complex, borane ammonia complex, borane di(tert-butyl)phosphine complex, borane dimethyl sulphide complex, borane methyl dodecyl sulphide complex, borane dimethylamine complex, borane diphenylphosphine complex, borane isoamylsulphide complex, borane morpholine complex, borane N,N-diethylaniline complex, borane N,N-diisopropylethylamine complex, borane pyridine complex, borane tert-butylamine complex, borane triethylamine complex, borane trimethylamine complex, borane triphenylphosphine complex, 2-picoline borane complex, and tert-butyldimethylphosphine borane complex); a hydrosilane and a catalyst (such as PhMe₂SiH and an acenaphthylene triruthenium carbonyl cluster; PhSiH₃ and MoO₂Cl₂; and Ph₂SiH₂ and RhH(CO)(PPh₃)₃); and tetrabutylammonium borohydride. The most preferred reducing agent is a borane complex, preferably borane-tetrahydrofuran complex. The group X is preferably chosen from an alkoxy group such as methoxy, ethoxy or t-butyloxy; an arylalkoxy group such as benzyloxy or p-methoxybenzyloxy; a halide group such as chloro, bromo or iodo; or an acyloxy group such as methoxycarbonyl, ethoxycarbonyl or benzoyl. The group X is preferably benzyloxy. The reaction is preferably carried out in an organic solvent such as tetrahydrofuran. The reaction temperature is preferably higher than 25° C., more preferably higher than 40° C. Most preferably the reaction is performed at reflux temperature in tetrahydrofuran. Preferably a reducing agent (preferably borane-tetrahydrofuran complex) is added to a solution of amido cyclohexanol (VIII). Preferably a solution of a reducing agent (preferably a solution of borane-tetrahydrofuran complex, more preferably a THF solution of borane-tetrahydrofuran complex) is added to a solution of amido cyclohexanol (VIII). Preferably amino cyclohexanol (IX) is isolated in high purity by selective extraction using a hydrocarbon solvent such as cyclohexene, toluene or xylene, preferably toluene. Preferably amino cyclohexanol (IX) is obtained from amido cyclohexanol (VIII) in a yield of 55%, 60%, 66%, 70%, 75%, 80% or more.

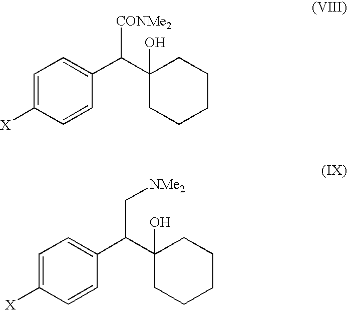

According to a sixth aspect of the invention there is provided a process for the preparation of O-desmethylvenlafaxine (I) or a pharmaceutically acceptable salt thereof, preferably the succinate or fumarate salt, wherein the process comprises a step according to the fifth aspect of the invention.

In a preferred embodiment of the invention, there is provided a process comprising one, two or all three steps of the first, third and fifth aspects of the invention.

According to a seventh aspect of the invention there is provided a process for the preparation of O-desmethylvenlafaxine (I) or a pharmaceutically acceptable salt thereof, preferably the succinate or fumarate salt, wherein the process comprises:
(a) reacting acid (VI) with dimethylamine or a salt thereof to form acetamide (VII),
(b) reacting acetamide (VII) with a base and cyclohexanone to form amido cyclohexanol (VIII), and
(c) reducing amido cyclohexanol (VIII) with a reducing agent to form amino cyclohexanol (IX),
   wherein the group X is any group capable of being converted into a hydroxyl group. The group X is preferably chosen from an alkoxy group such as methoxy, ethoxy or t-butyloxy; an arylalkoxy group such as benzyloxy or p-methoxybenzyloxy; a halide group such as chloro, bromo or iodo; or an acyloxy group such as methoxycarbonyl, ethoxycarbonyl or benzoyl. The group X is preferably benzyloxy.

The conversion of the substituted phenyl acetic acid (VI) to the corresponding acetamide (VII) in step (a) according to the seventh aspect of the invention is preferably carried out without isolating the intermediate acid chloride. Preferably step (a) is performed with thionyl chloride. Preferably dimethylamine hydrochloride is used in step (a).

The base in step (b) is preferably strong, non-nucleophilic, bulky and/or sterically hindered. Preferably the base is less nucleophilic and/or more bulky and/or more sterically hindered than n-BuLi and/or LDA. Preferred bases are lithium hexamethyl disilazide (LHMDS), potassium tertiary butoxide, NaNH₂, DBU (1,8-diazabicyclo[5.4.0]undec-7-ene), and DBN (1,5-diazabicyclo[4.3.0]non-5-ene). The most preferred base is lithium hexamethyl disilazide (LHMDS).

Preferred reducing agents in step (c) are a borane complex (such as borane-tetrahydrofuran complex, methyl 6-morpholinohexyl sulphide borane complex, borane 1,2-bis(tert-butylthio)ethane complex, borane 4-methylmorpholine complex, borane ammonia complex, borane di(tert-butyl)phosphine complex, borane dimethyl sulphide complex, borane methyl dodecyl sulphide complex, borane dimethylamine complex, borane diphenylphosphine complex, borane isoamylsulphide complex, borane morpholine complex, borane N,N-diethylaniline complex, borane N,N-diisopropylethylamine complex, borane pyridine complex, borane tert-butylamine complex, borane triethylamine complex, borane trimethylamine complex, borane triphenylphosphine complex, 2-picoline borane complex, and tert-butyldimethylphosphine borane complex); a hydrosilane and a catalyst (such as PhMe₂SiH and an acenaphthylene triruthenium carbonyl cluster; PhSiH₃ and MoO₂Cl₂; and Ph₂SiH₂ and RhH(CO)(PPh₃)₃); and tetrabutylammonium borohydride. The most preferred reducing agent is a borane complex, preferably borane-tetrahydrofuran complex. Preferably a reducing agent (preferably borane-tetrahydrofuran complex) is added to a solution of amido cyclohexanol (VIII). Preferably a solution of a reducing agent (preferably a solution of borane-tetrahydrofuran complex, more preferably a THF solution of borane-tetrahydrofuran complex) is added to a solution of amido cyclohexanol (VIII).

In step (c), cyclohexanol (IX) is preferably isolated by extraction using a hydrocarbon solvent such as cyclohexene, toluene or xylene, preferably toluene.

The process according to the seventh aspect of the invention is outlined in the scheme in FIG. 2.

Amino cyclohexanol (IX) can be converted to ODV (I) by conversion of group X to a hydroxyl group:

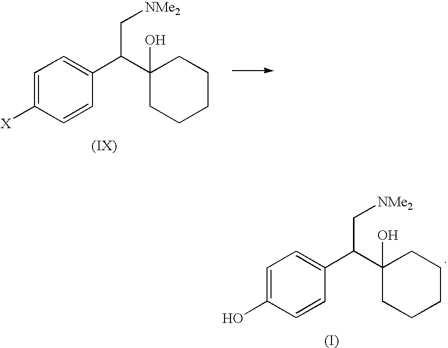

In a preferred embodiment of the present invention, the group X is a benzyloxy group and is typically converted to the corresponding hydroxyl compound by catalytic hydrogenolysis in the presence of Pd/C.

Preferably ODV (I) is obtained from amino cyclohexanol (IX) in a yield of 80%, 87%, 90%, 95% or more. Preferably ODV (I) is obtained from acid (VI) in a yield of 25%, 30%, 40%, 50%, 60%, 70% or more.

The processes of the present invention are capable of providing acetamide (VII), amido cyclohexanol (VIII), amino cyclohexanol (IX), O-desmethylvenlafaxine (I) and pharmaceutically acceptable O-desmethylvenlafaxine salts, in consistent chemical purity irrespective of the scale of preparation. Preferably acetamide (VII), amido cyclohexanol (VIII), amino cyclohexanol (IX), O-desmethylvenlafaxine (I), or a pharmaceutically acceptable O-desmethylvenlafaxine salt, is obtained in an HPLC purity of 95%, 98%, 99%, 99.5%, 99.7%, 99.9% or more.

Preferably the processes of the present invention are carried out on an industrial scale, preferably to manufacture acetamide (VII), amido cyclohexanol (VIII), amino cyclohexanol (IX), O-desmethylvenlafaxine (I), or a pharmaceutically acceptable O-desmethylvenlafaxine salt, in batches of 0.1 kg, 0.5 kg, 1 kg, 5 kg, 10 kg, 50 kg, 100 kg, 500 kg or more.

ODV (I) exists as enantiomers and the present invention includes the racemic mixture as well as stereoisomerically pure forms of ODV (I). The term ‘ODV free base’ as used herein refers to racemic mixtures and stereoisomerically pure forms of ODV free base, unless otherwise indicated. The term ‘stereoisomerically pure’ refers to compounds, which are comprised of a greater proportion of the desired isomer than that of the optical antipode. Stereoisomerically pure ODV free base is generally made up of at least 90% of the desired isomer based upon 100% total weight of ODV free base.

An eighth aspect of the invention provides O-desmethylvenlafaxine (I) or a pharmaceutically acceptable salt thereof, obtained by a process according to any one of the first to seventh aspects of the present invention. Preferably the pharmaceutically acceptable salt is O-desmethylvenlafaxine succinate or fumarate salt.

A ninth aspect of the invention provides a pharmaceutical composition comprising O-desmethylvenlafaxine (I) or a pharmaceutically acceptable salt thereof according to the eighth aspect of the present invention.

The dosage form can be a solution or suspension form, but is preferably solid and comprises one or more conventional pharmaceutically acceptable excipient(s). Preferred dosage forms in accordance with the invention include tablets, capsules and the like. Tablets can be prepared by conventional techniques, including direct compression, wet granulation and dry granulation. Capsules are generally formed from a gelatine material and can include a conventionally prepared granulate of excipients and adduct or solvate in accordance with the invention.

A tenth aspect of the invention provides use of O-desmethylvenlafaxine (I) or a pharmaceutically acceptable salt thereof, according to the eighth aspect of the present invention, for the preparation of a medicament for the treatment or prevention of depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.

An eleventh aspect of the invention provides a method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease, the method comprising administering a therapeutically or prophylactically effective amount of O-desmethylvenlafaxine (I) or a pharmaceutically acceptable salt thereof, according to the eighth aspect of the present invention, to a patient in need thereof. Preferably the patient is a human.

### BRIEF DESCRIPTION OF THE DRAWINGS

The present invention will now be described by way of example with reference to the accompanying drawings in which:

FIG. 1 is a schematic representation of a prior art process for the preparation of O-desmethylvenlafaxine (I).

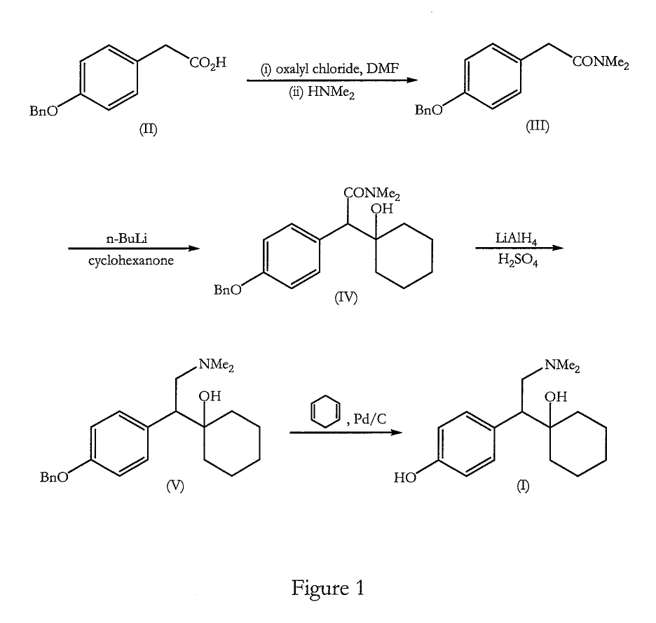

FIG. 2 is a schematic representation of a process according to the present invention.

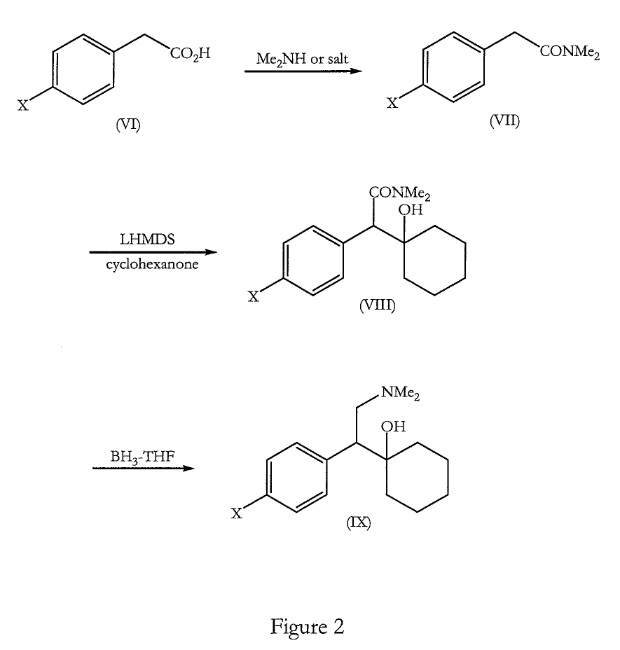

FIG. 3 is a schematic representation of a particularly preferred embodiment of a process according to the present invention.

### DETAILED DESCRIPTION OF THE INVENTION

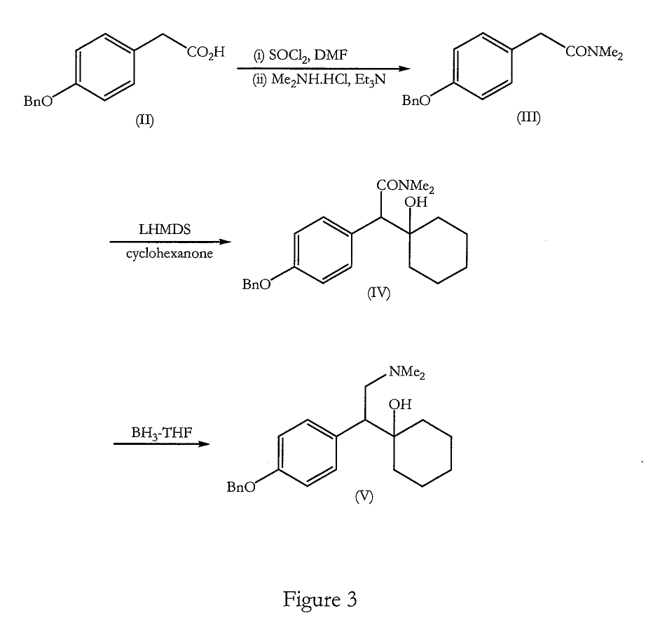

A particularly preferred embodiment of a process according to the present invention is illustrated in the scheme in FIG. 3.

Acetamide (III) can be prepared from acid (II) via the acid chloride. The process is simplified by obtaining acetamide (III) without isolating the intermediate acid chloride. One advantage of the present invention is use of thionyl chloride instead of oxalyl chloride which is used in the prior art (U.S. Pat. No. 4,535,186 and U.S. Pat. No. 4,761,501). Use of oxalyl chloride can lead to an emission of carbon monoxide, which is hazardous especially on plant scale. Use of thionyl chloride on the other hand is much safer. Moreover, thionyl chloride is much less expensive than oxalyl chloride and therefore the process becomes more cost efficient by using thionyl chloride. Another advantage of the present invention is use of commercially easily available dimethylamine hydrochloride in combination with triethylamine (preferably about 3 equivalents each with respect to acid (II)).

Another advantage of the present invention is the improved condensation of the lithium salt of acetamide (III) with cyclohexanone to obtain amido cyclohexanol (IV). The prior art (U.S. Pat. No. 4,535,186 and U.S. Pat. No. 4,761,501) describes a method of condensing cyclohexanone with 4-substituted phenyl acetamide employing n-butyl lithium or lithium diisopropyl amide (LDA). These reagents are highly sensitive to moisture and air, unsafe, potentially hazardous, and expensive on plant scale. Moreover the yield according to U.S. Pat. No. 4,535,186 and U.S. Pat. No. 4,761,501 is low and does not exceed 50%. The present invention discloses use of the relatively safe, economical, user-friendly, non-nucleophilic silyl base lithium hexamethyl disilazide (LHMDS) in organic solvents such as dry tetrahydrofuran, toluene, diisopropyl ether, methyl t-butyl ether, diethyl ether etc. The reaction involving LHMDS does not require stringent anhydrous conditions as are required for n-butyl lithium and LDA. The use of LHMDS has a significant impact on up-scaling of the process besides improvement in yield and purity.

Another advantage of the present invention is a safe process for the reduction of amido cyclohexanol (IV) to amino cyclohexanol (V). The prior art (U.S. Pat. No. 4,535,186 and U.S. Pat. No. 4,761,501) describes the reduction of amido cyclohexanol (IV) with aluminium hydride generated in situ by reaction of lithium aluminium hydride and conc. sulphuric acid. Handling of these reagents on plant scale is very difficult and hazardous. The present invention avoids use of potentially hazardous reagents such as lithium aluminium hydride and conc. sulphuric acid by employing safer reagents. The present invention describes a method involving use of borane-tetrahydrofuran complex for the reduction of amido cyclohexanol (IV) to amino cyclohexanol (V). The use of borane-tetrahydrofuran complex in accordance with the present invention was modified to make it amenable to large scale production. In particular, in a preferred embodiment of the present invention borane-tetrahydrofuran complex is added to a solution of amido cyclohexanol (IV). Moreover, the present invention offers a simple work-up procedure with improved yield and quality. Amino cyclohexanol (V) can be isolated from the reaction mixture by selective extraction with toluene. This selective extraction affords high quality amino cyclohexanol (V) with minimum contamination by process impurities.

The amino cyclohexanol (V) can be converted into ODV free base (I) by debenzylation in the presence of Pd/C in alcohol, following standard reaction conditions. The following paragraphs enumerated consecutively from 1 through 42 provide for various aspects of the present invention. In one embodiment, in a first paragraph (1), the present invention provides:

1. A process for the preparation of acetamide (VII), comprising reacting acid (VI) with thionyl chloride and dimethylamine or a salt thereof, wherein the group X is any group capable of being converted into a hydroxyl group:

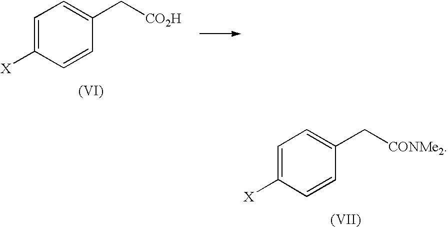

2. A process according to paragraph 1, wherein the dimethylamine is used as the hydrochloride salt.
3. A process according to paragraph 1 or 2, wherein the process is carried out without isolating the intermediate acid chloride.
4. A process for the preparation of amido cyclohexanol (VIII), comprising reacting acetamide (VII) with a base and cyclohexanone, wherein the group X is any group capable of being converted into a hydroxyl group:

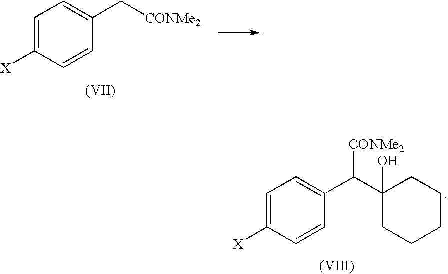

5. A process according to paragraph 4, wherein the base is lithium hexamethyl disilazide (LHMDS), potassium tertiary butoxide, NaNH₂, DBU (1,8-diazabicyclo[5.4.0]undec-7-ene), or DBN (1,5-diazabicyclo[4.3.0]non-5-ene).
6. A process according to paragraph 5, wherein the base is lithium hexamethyl disilazide (LHMDS).
7. A process for the preparation of amino cyclohexanol (IX), comprising reducing amido cyclohexanol (VIII) with a reducing agent, wherein the group X is any group capable of being converted into a hydroxyl group:

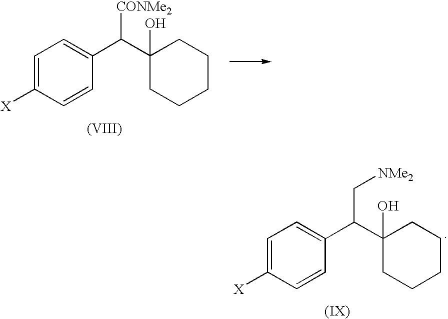

8. A process according to paragraph 7, wherein the reducing agent is a borane complex; a hydrosilane and a catalyst; or tetrabutylammonium borohydride.
9. A process according to paragraph 8, wherein the reducing agent is borane-tetrahydrofuran complex.
10. A process according to any one of paragraphs 7 to 9, wherein amino cyclohexanol (IX) is isolated by extraction using a hydrocarbon solvent.
11. A process according to paragraph 10, wherein the hydrocarbon solvent is cyclohexene, toluene or xylene.
12. A process according to paragraph 11, wherein the hydrocarbon solvent is toluene.
13. A process according to any one of paragraphs 7 to 12, wherein a reducing agent is added to a solution of amido cyclohexanol (VIII).
14. A process according to paragraph 13, wherein borane-tetrahydrofuran complex is added to a solution of amido cyclohexanol (VIII).
15. A process for the preparation of O-desmethylvenlafaxine (I) or a pharmaceutically acceptable salt thereof, comprising a process step according to any one of the preceding paragraphs.
16. A process for the preparation of O-desmethylvenlafaxine (I) or a pharmaceutically acceptable salt thereof, wherein the process comprises:
(a) reacting acid (VI) with dimethylamine or a salt thereof to form acetamide (VII):

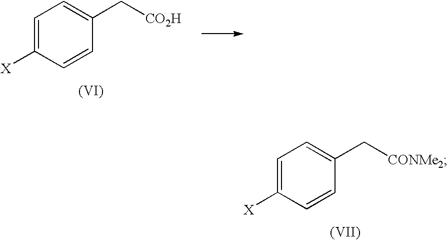

(b) reacting acetamide (VII) with a base and cyclohexanone to form amido cyclohexanol (VIII):

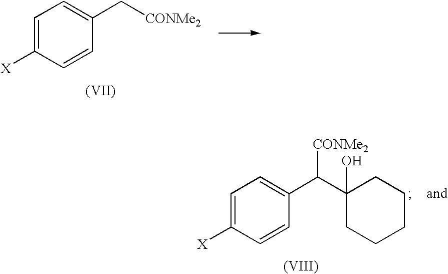

(c) reducing amido cyclohexanol (VIII) with a reducing agent to form amino cyclohexanol (IX):

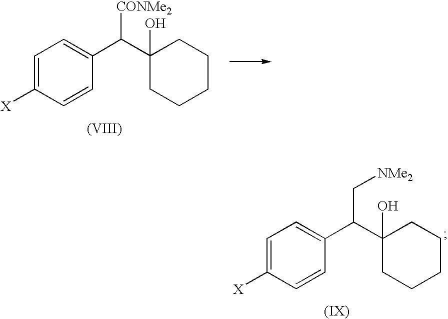

wherein the group X is any group capable of being converted into a hydroxyl group.
17. A process according to paragraph 16, wherein an acid chloride is formed in step (a), but not isolated.
18. A process according to paragraph 16 or 17, wherein step (a) is performed with thionyl chloride.
19. A process according to any one of paragraphs 16 to 18, wherein dimethylamine hydrochloride is used in step (a).
20. A process according to any one of paragraphs 16 to 19, wherein the base in step (b) is lithium hexamethyl disilazide (LHMDS), potassium tertiary butoxide, NaNH₂, DBU (1,8-diazabicyclo[5.4.0]undec-7-ene), or DBN (1,5-diazabicyclo[4.3.0]non-5-ene).
21. A process according to paragraph 20, wherein the base in step (b) is lithium hexamethyl disilazide (LHMDS).
22. A process according to any one of paragraphs 16 to 21, wherein the reducing agent is a borane complex; a hydrosilane and a catalyst; or tetrabutylammonium borohydride.
23. A process according to paragraph 22, wherein the reducing agent is borane-tetrahydrofuran complex.
24. A process according to any one of paragraphs 16 to 23, wherein in step (c) cyclohexanol (IX) is isolated by extraction using a hydrocarbon solvent.
25. A process according to paragraph 24, wherein the hydrocarbon solvent is cyclohexene, toluene or xylene.
26. A process according to paragraph 25, wherein the hydrocarbon solvent is toluene.
27. A process according to any one of paragraphs 16 to 26, wherein in step (c) a reducing agent is added to a solution of amido cyclohexanol (VIII).
28. A process according to paragraph 27, wherein in step (c) borane-tetrahydrofuran complex is added to a solution of amido cyclohexanol (VIII).
29. A process according to any one of paragraphs 7 to 26, wherein amino cyclohexanol (IX) is converted to O-desmethylvenlafaxine (I) or a pharmaceutically acceptable salt thereof:

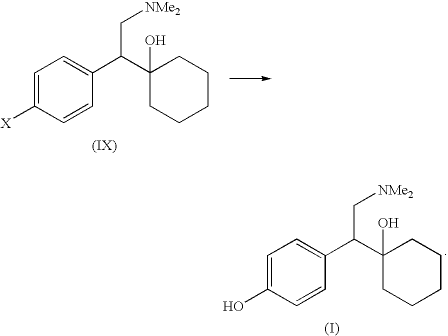

30. A process according to any one of paragraphs 15 to 29, wherein O-desmethylvenlafaxine succinate or fumarate salt is obtained.
31. A process according to any one of the preceding paragraphs, wherein the group X is chosen from an alkoxy group, an arylalkoxy group, a halide group or an acyloxy group.
32. A process according to paragraph 31, wherein the alkoxy group is selected from methoxy, ethoxy or t-butyloxy; the arylalkoxy group is selected from benzyloxy or p-methoxybenzyloxy; the halide group is selected from chloro, bromo or iodo; or the acyloxy group is selected from methoxycarbonyl, ethoxycarbonyl or benzoyl.
33. A process according to paragraph 32, wherein the group X is benzyloxy.
34. A process according to any one of the preceding paragraphs, wherein O-desmethylvenlafaxine (I) is obtained from acid (VI) in a yield of 25% or more.
35. A process according to any one of the preceding paragraphs, wherein O-desmethylvenlafaxine (I) is obtained in an HPLC purity of 95% or more.
36. A process according to any one of the preceding paragraphs, wherein the process is carried out on an industrial scale.
37. O-Desmethylvenlafaxine (I) or a pharmaceutically acceptable salt thereof, obtained by a process according to any one of paragraphs 1 to 36.
38. A compound according to paragraph 37, wherein the pharmaceutically acceptable salt is O-desmethylvenlafaxine succinate or fumarate salt.
39. A pharmaceutical composition comprising O-desmethylvenlafaxine (I) or a pharmaceutically acceptable salt thereof, as in paragraph 37 or 38.
40. Use of O-desmethylvenlafaxine (I) or a pharmaceutically acceptable salt thereof as in paragraph 37 or 38 for the preparation of a medicament for the treatment or prevention of depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.
41. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease, comprising administering a therapeutically or prophylactically effective amount of O-desmethylvenlafaxine (I) or a pharmaceutically acceptable salt thereof as in paragraph 37 or 38 to a patient in need thereof.
42. A method according to paragraph 41, wherein the patient is a human.

### EXAMPLES

The details of the invention, its objects and advantages are explained hereunder in greater detail in the following non-limiting examples.

### Example 1

### Preparation of 4-benzyloxy-N,N-dimethyl-benzeneacetamide (III)

4-Benzyloxyphenyl acetic acid (100 g, 0.41 mol) was reacted with thionyl chloride (45 ml, 0.62 mol) in refluxing dichloromethane in the presence of a catalytic amount of N,N-dimethyl formamide (one drop) for 6 hours. The acid chloride solution was added to a well stirred mixture of dimethylamine hydrochloride (101 g, 1.24 mol) and triethylamine (174 ml, 1.24 mol) in dichloromethane whilst maintaining the temperature below 5° C. After completion of the addition, stirring was continued for a further 1 hour at 5-10° C. The reaction was quenched by adding water (1000 ml) and the organic layer was separated. The aqueous layer was extracted with dichloromethane (3×500 ml) and the combined extracts were washed with water (2×500 ml). The organic layer was dried over anhydrous sodium sulphate and concentration of the organic layer afforded a residue. To the residue obtained, hexane (700 ml) was added and stirred for 1 hour at 25-30° C. It was then filtered to obtain acetamide (III) as an off-white solid. The structure of the product was confirmed by ¹H-NMR. Weight of the product=100 g; molar yield=90%.

### Example 2

### Preparation of 1-[(4-benzyloxyphenyl)-dimethylaminocarbonyl-methyl]cyclohexanol (IV)

A solution of lithium hexamethyl disilazide (LHMDS) in tetrahydrofuran (388.5 ml, 0.46 mol) was added to a well stirred solution of acetamide (III) (50 g, 0.185 mol) in dry tetrahydrofuran (1000 ml) maintaining the temperature at −60° C. to −70° C. The mixture was stirred for 15 minutes. Cyclohexanone (23 ml, 0.223 mol) was added maintaining the temperature at −60° C. to −70° C. After completion of the reaction, a saturated aqueous solution of ammonium chloride was added. Dilute hydrochloric acid was added to adjust the pH to 4.0 and the mixture was extracted with ethyl acetate (3×150 ml). The combined organic extracts were washed with water and dried over anhydrous sodium sulphate. Concentration of the extracts afforded the desired amide (IV) as an off-white solid. The structure of the product was confirmed by ¹H-NMR. Weight of the product=56 g; molar yield=82%.

### Example 3

### Preparation of 1-[1-(4-benzyloxyphenyl)-2-(dimethylamino)ethyl]cyclohexanol (V)

Borane-tetrahydrofuran solution (68 ml, 0.068 mol) was added to a solution of amide (IV) (5 g, 0.0136 mol) in tetrahydrofuran (50 ml) at 0° C. The reaction mixture was stirred for 20 minutes at 0° C. and then heated to reflux for 3 hours. After completion of the reaction, 10% hydrochloric acid (25 ml) was added and heated to reflux for 3 hours. The reaction mixture was allowed to cool to 10° C. and then neutralized by adding 10% aqueous sodium hydroxide. The reaction medium was concentrated to remove tetrahydrofuran, water (50 ml) was added and the mixture was extracted with toluene (3×100 ml). The combined extracts were dried over anhydrous sodium sulphate and concentrated. Hexane (100 ml) was added to the residue and stirred for 15 minutes. It was filtered to obtain the desired amine (V) as an off-white solid. The structure of the product was confirmed by ¹H-NMR. Weight of the product=3.2 g; molar yield=66%.

### Example 4

### Preparation of ODV Free Base (I)

Amine (V) (2 g) was debenzylated in the presence of hydrogen gas over 20% Pd/C (0.2 g, 10% w/w) in ethanol (50 ml) at atmospheric pressure and at 25° C. After completion of the reaction, the mixture was filtered through Celite® to remove catalyst. The filtrate was concentrated to obtain a residue. Hexane (50 ml) was added to the residue and stirred for 30 minutes. It was then filtered to obtain ODV free base (I) as an off-white solid. The structure of the product was confirmed by ¹H-NMR. Weight of the product=1.3 g; molar yield=87%.

### Example 5

### Preparation of ODV Free Base (I) on an Industrial Scale

The processes described in examples 1 to 4 were carried out on an industrial scale producing ODV free base (I) in the following amounts and HPLC purities: 150 g with an HPLC purity of 99.66%, 300 g with an HPLC purity of 99.70%, and 12 kg with an HPLC purity of 99.71%.

It will be understood that the present invention has been described above by way of example only. The examples are not intended to limit the scope of the invention. Various modifications and embodiments can be made without departing from the scope and spirit of the invention, which is defined by the following claims only.

What is claimed is:

1. A process for the preparation of acetamide (VII), comprising reacting acid (VI) with thionyl chloride and dimethylamine or a salt thereof, wherein the group X is any group capable of being converted into a hydroxyl group:

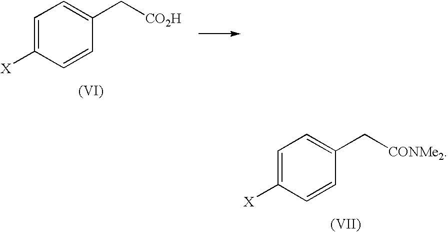

2. A process according to claim 1, wherein:
(i) the dimethylamine is used as the hydrochloride salt; and/or
(ii) the process is carried out without isolating the intermediate acid chloride.

3. A process for the preparation of amido cyclohexanol (VIII), comprising reacting acetamide (VII) with a base and cyclohexanone, wherein the group X is any group capable of being converted into a hydroxyl group:

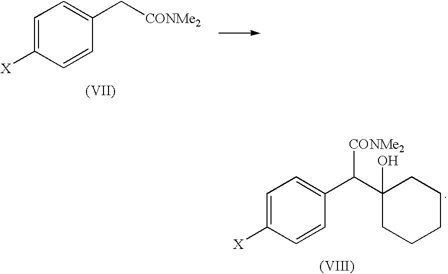

4. A process according to claim 3, wherein:
(i) the base is lithium hexamethyl disilazide (LHMDS), potassium tertiary butoxide, NaNH₂, DBU (1,8-diazabicyclo[5.4.0]undec-7-ene), or DBN (1,5-diazabicyclo[4.3.0]non-5-ene); and/or
(ii) the base is lithium hexamethyl disilazide (LHMDS).

5. A process for the preparation of amino cyclohexanol (IX), comprising reducing amido cyclohexanol (VIII) with a reducing agent, wherein the group X is any group capable of being converted into a hydroxyl group:

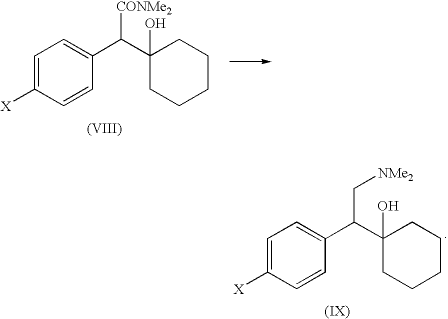

6. A process according to claim 5, wherein:
(i) the reducing agent is a borane complex; a hydrosilane and a catalyst; or tetrabutylammonium borohydride; and/or
(ii) the reducing agent is borane-tetrahydrofuran complex; and/or
(iii) amino cyclohexanol (IX) is isolated by extraction using a hydrocarbon solvent; and/or
(iv) amino cyclohexanol (IX) is isolated by extraction using cyclohexene, toluene or xylene; and/or
(v) amino cyclohexanol (IX) is isolated by extraction using toluene; and/or
(vi) the reducing agent is added to a solution of amido cyclohexanol (VIII); and/or
(vii) borane-tetrahydrofuran complex is added to a solution of amido cyclohexanol (VIII).

7. A process for the preparation of O-desmethylvenlafaxine (I) or a pharmaceutically acceptable salt thereof, comprising a process step according to claim 1.

8. A process for the preparation of O-desmethylvenlafaxine (I) or a pharmaceutically acceptable salt thereof, comprising a process step according to claim 3.

9. A process for the preparation of O-desmethylvenlafaxine (I) or a pharmaceutically acceptable salt thereof, comprising a process step according to claim 5.

10. A process for the preparation of O-desmethylvenlafaxine (I) or a pharmaceutically acceptable salt thereof, wherein the process comprises:
reacting acid (VI) with dimethylamine or a salt thereof to form acetamide (VII): 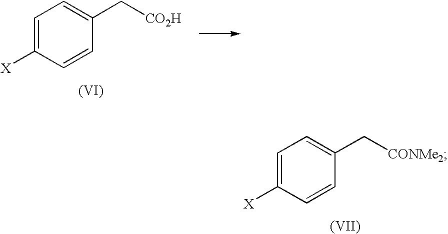
reacting acetamide (VII) with a base and cyclohexanone to form amido cyclohexanol (VIII): 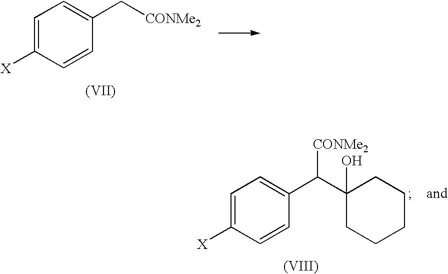
reducing amido cyclohexanol (VIII) with a reducing agent to form amino cyclohexanol (IX): 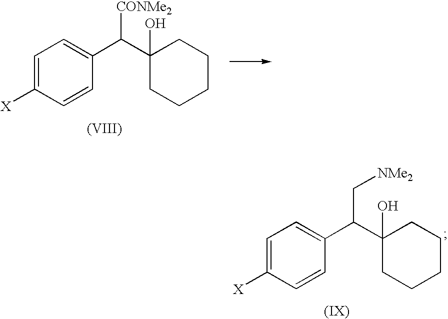
wherein the group X is any group capable of being converted into a hydroxyl group.

11. A process according to claim 10, wherein:
(i) an acid chloride is formed in step (a), but not isolated; and/or
(ii) step (a) is performed with thionyl chloride; and/or
(iii) dimethylamine hydrochloride is used in step (a); and/or
(iv) the base in step (b) is lithium hexamethyl disilazide (LHMDS), potassium tertiary butoxide, NaNH₂, DBU (1,8-diazabicyclo[5.4.0]undec-7-ene), or DBN (1,5-diazabicyclo[4.3.0]non-5-ene); and/or
(v) the base in step (b) is lithium hexamethyl disilazide (LHMDS); and/or
(vi) the reducing agent in step (c) is a borane complex; a hydrosilane and a catalyst; or tetrabutylammonium borohydride; and/or
(vii) the reducing agent in step (c) is borane-tetrahydrofuran complex; and/or
(viii) in step (c) cyclohexanol (IX) is isolated by extraction using a hydrocarbon solvent; and/or
(ix) in step (c) cyclohexanol (IX) is isolated by extraction using cyclohexene, toluene or xylene; and/or
(x) in step (c) cyclohexanol (IX) is isolated by extraction using toluene; and/or
(xi) in step (c) the reducing agent is added to a solution of amido cyclohexanol (VIII); and/or
(xii) in step (c) borane-tetrahydrofuran complex is added to a solution of amido cyclohexanol (VIII).

12. A process according to claim 10, wherein amino cyclohexanol (IX) is converted to O-desmethylvenlafaxine (I) or a pharmaceutically acceptable salt thereof:

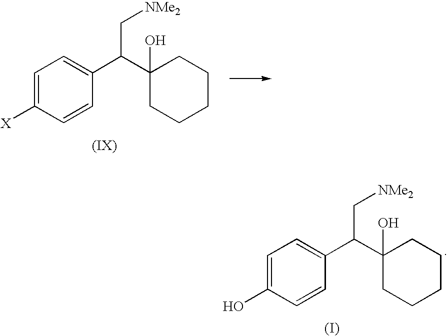

13. A process according to claim 10, wherein O-desmethylvenlafaxine succinate or fumarate salt is obtained.

14. A process according to claim 1, wherein:
(i) the group X is chosen from an alkoxy group, an arylalkoxy group, a halide group or an acyloxy group; and/or
(ii) the group X is chosen from an alkoxy group, an arylalkoxy group, a halide group or an acyloxy group, and wherein the alkoxy group is selected from methoxy, ethoxy or t-butyloxy; the arylalkoxy group is selected from benzyloxy or p-methoxybenzyloxy; the halide group is selected from chloro, bromo or iodo; or the acyloxy group is selected from methoxycarbonyl, ethoxycarbonyl or benzoyl; and/or
(iii) the group X is benzyloxy.

15. A process according to claim 3, wherein:
(i) the group X is chosen from an alkoxy group, an arylalkoxy group, a halide group or an acyloxy group; and/or
(ii) the group X is chosen from an alkoxy group, an arylalkoxy group, a halide group or an acyloxy group, and wherein the alkoxy group is selected from methoxy, ethoxy or t-butyloxy; the arylalkoxy group is selected from benzyloxy or p-methoxybenzyloxy; the halide group is selected from chloro, bromo or iodo; or the acyloxy group is selected from methoxycarbonyl, ethoxycarbonyl or benzoyl; and/or
(iii) the group X is benzyloxy.

16. A process according to claim 5, wherein:
(i) the group X is chosen from an alkoxy group, an arylalkoxy group, a halide group or an acyloxy group; and/or
(ii) the group X is chosen from an alkoxy group, an arylalkoxy group, a halide group or an acyloxy group, and wherein the alkoxy group is selected from methoxy, ethoxy or t-butyloxy; the arylalkoxy group is selected from benzyloxy or p-methoxybenzyloxy; the halide group is selected from chloro, bromo or iodo; or the acyloxy group is selected from methoxycarbonyl, ethoxycarbonyl or benzoyl; and/or
(iii) the group X is benzyloxy.

17. A process according to claim 10, wherein:
(i) the group X is chosen from an alkoxy group, an arylalkoxy group, a halide group or an acyloxy group; and/or
(ii) the group X is chosen from an alkoxy group, an arylalkoxy group, a halide group or an acyloxy group, and wherein the alkoxy group is selected from methoxy, ethoxy or t-butyloxy; the arylalkoxy group is selected from benzyloxy or p-methoxybenzyloxy; the halide group is selected from chloro, bromo or iodo; or the acyloxy group is selected from methoxycarbonyl, ethoxycarbonyl or benzoyl; and/or
(iii) the group X is benzyloxy; and/or
(iv) O-desmethylvenlafaxine (I) is obtained from acid (VI) in a yield of 25% or more; and/or
(v) O-desmethylvenlafaxine (I) is obtained in an HPLC purity of 95% or more; and/or
(vi) the process is carried out on an industrial scale.

18. O-Desmethylvenlafaxine (I) or a pharmaceutically acceptable salt thereof, obtained by a process according to claim 7.

19. O-Desmethylvenlafaxine (I) or a pharmaceutically acceptable salt thereof, obtained by a process according to claim 8.

20. O-Desmethylvenlafaxine (I) or a pharmaceutically acceptable salt thereof, obtained by a process according to claim 9.

21. O-Desmethylvenlafaxine (I) or a pharmaceutically acceptable salt thereof, obtained by a process according to claim 10.

22. A compound according to claim 21, wherein the pharmaceutically acceptable salt is O-desmethylvenlafaxine succinate or fumarate salt.

23. A pharmaceutical composition comprising O-desmethylvenlafaxine (I) or a pharmaceutically acceptable salt thereof as claimed in claim 21.

24. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease, comprising administering a therapeutically or prophylactically effective amount of O-desmethylvenlafaxine (I) or a pharmaceutically acceptable salt thereof as claimed in claim 21 to a patient in need thereof.

25. A method according to claim 24, wherein the patient is a human.
